(19) United States
(12) Patent Application Publication
(10) Pub. No.: US 2022/0319699 A1
(43) Pub. Date: Oct. 6, 2022

(54) SYSTEM AND METHOD FOR GENERATING A TOXICOLOGICAL AILMENT NOURISHMENT PROGRAM

(71) Applicant: KPN INNOVATIONS, LLC., Lakewood, CO (US)

(72) Inventor: Kenneth Neumann, Lakewood, CO (US)

(73) Assignee: KPN INNOVATIONS, LLC., Lakewood, CO (US)

(21) Appl. No.: 17/221,430

(22) Filed: Apr. 2, 2021

(51) Int. Cl. G16H 50/20; G16B 40/00; G06N 20/20

(52) U.S. Cl. CPC G16H 50/20; G16B 40/00; G06N 20/20

(57) ABSTRACT

A system for generating a toxicological ailment nourishment program includes a computing device configured to obtain a toxicological indicator, identify a toxicological profile as a function of the toxicological indicator, wherein identifying further comprises determining at least a xenobiotic as a function of the toxicological indicator, obtaining an exposure input, and identifying the toxicological profile as a function of the at least a xenobiotic and the exposure input using a profile machine-learning model, determine an edible as a function of the toxicological profile, and generate a nourishment program as a function of the edible.

### FIELD OF THE INVENTION

The present invention generally relates to the field of artificial intelligence. In particular, the present invention is directed to a system and method for generating a toxicological ailment nourishment program.

### BACKGROUND

Current edible suggestion systems do not account for the presence of one or more xenobiotics in an individual. This leads to inefficiency of an edible suggestion system and a poor nutrition plan for the individual. This is further complicated by a lack of uniformity of nutritional plans, which results in dissatisfaction of individuals.

### SUMMARY OF THE DISCLOSURE

In an aspect, a system for generating a toxicological ailment nourishment program includes a computing device configured to obtain a toxicological indicator, identify a toxicological profile as a function of the toxicological indicator, wherein identifying further comprises determining at least a xenobiotic as a function of the toxicological indicator, obtaining an exposure input, and identifying the toxicological profile as a function of the at least a xenobiotic and the exposure input using a profile machine-learning model, determine an edible as a function of the toxicological profile, and generate a nourishment program as a function of the edible.

In another aspect, a method for generating a toxicological ailment nourishment program includes obtaining, by a computing device, a toxicological indicator, identifying, by the computing device, a toxicological profile as a function of the toxicological indicator, wherein identifying further comprises determining at least a xenobiotic as a function of the toxicological indicator, obtaining an exposure input, and identifying the toxicological profile as a function of the at least a xenobiotic and the exposure input using a profile machine-learning model, determining, by the computing device, an edible as a function of the toxicological profile, and generating, by the computing device, a nourishment program as a function of the edible.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating a toxicological ailment nourishment program;

FIG. 2 is a block diagram of an exemplary embodiment of a chemical interaction according to an embodiment of the invention;

FIG. 3 is a block diagram of an exemplary embodiment of an elimination element according to an embodiment of the invention;

FIG. 4 is a block diagram of an exemplary embodiment of a hormetic element according to an embodiment of the invention;

FIG. 5 is a block diagram of an exemplary embodiment of a machine-learning module;

FIG. 6 is a process flow diagram illustrating an exemplary embodiment of a method of generating a toxicological ailment nourishment program; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a toxicological ailment nourishment program. In an embodiment, this disclosure obtains a toxicological indicator. Aspects of the present disclosure can be used to identify a toxicological profile as a function of a xenobiotic and an exposure input. This is so, at least in part, because this disclosure utilizes a profile machine-learning model. Aspects of the present disclosure can also be used to determine an edible. Aspects of the present disclosure allow for generating a nourishment program as a function of the edible. Exemplary embodiments illustrating aspects of the present disclosure are described below in the context of several specific examples.

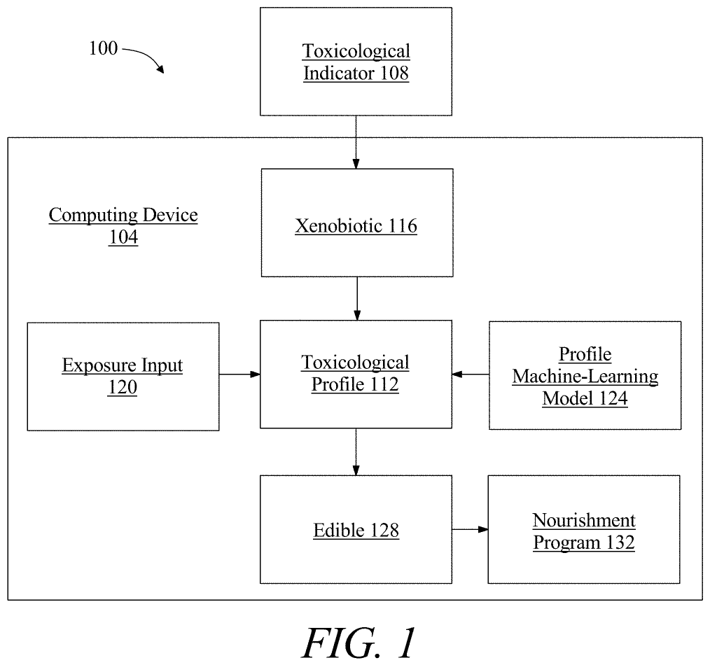

Referring now to FIG. 1, an exemplary embodiment of a system 100 for generating a toxicological ailment nourishment program is illustrated. System includes a computing device 104. computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. computing device 104 may include a single computing device operating independently, or may include two or more computing device operating in concert, in parallel, sequentially or the like; two or more computing devices may be included together in a single computing device or in two or more computing devices. computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. computing device 104 may include but is not limited to, for example, a computing device or cluster of computing devices in a first location and a second computing device or cluster of computing devices in a second location. computing device 104 may include one or more computing devices dedicated to data storage, security, distribution of traffic for load balancing, and the like. computing device 104 may distribute one or more computing tasks as described below across a plurality of computing devices of computing device, which may operate in parallel, in series, redundantly, or in any other manner used for distribution of tasks or memory between computing devices. computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 is configured to obtain a toxicological indicator 108. As used in this disclosure a “toxicological indicator” is an element of data associated with an individual's biological system that denotes a health status of the individual, wherein a health status is a measure of the relative level of physical, social and/or behavioral well-being. In an embodiment, toxicological indicator 108 may denote one or more health statuses of an individual's nervous system, circulatory system, musculoskeletal system, respiratory system, endocrine system, integumentary system, lymphatic system, digestive system, urinary system, reproductive system, and the like thereof. In an embodiment, toxicological indicator 108 may include a mutation indicator. As used in this disclosure a “mutation indicator” is an element that denotes a mutation. For example, and without limitation mutation indicator may denote a cleavage of DNA and/or impingement on the reproduction of DNA. In an embodiment, and still referring to FIG. 1, toxicological indicator 108 may include a biological sample. As used in this disclosure a “biological sample” is one or more biological specimens collected from an individual. Biological sample may include, without limitation, exhalate, blood, sputum, urine, saliva, feces, semen, and other bodily fluids, as well as tissue. Toxicological indicator 108 may include a biological sampling device. Toxicological indicator 108 may include one or more biomarkers. As used in this disclosure a “biomarker” is a molecule and/or chemical that identifies the status of an individual's health system. As a non-limiting example, toxicological indicators may include, heavy metals, halogenated hydrocarbons, pesticides, venoms, radioactive materials, cytochrome p-450 enzymes, catechol-O-methyltransferases, nephrotoxic compounds, metallothioneins, heat stress proteins, and the like thereof. As a further non-limiting example, toxicological indicator 108 may include datum from one or more devices that collect, store, and/or calculate one or more lights, voltages, currents, sounds, chemicals, pressures, and the like thereof that are associated with the individual's health status. For example, and without limitation a device may include a magnetic resonance imaging device, magnetic resonance spectroscopy device, x-ray spectroscopy device, computerized tomography device, ultrasound device, electroretinogram device, electrocardiogram device, ABER sensor, mass spectrometer, and the like thereof.

Still referring to FIG. 1, computing device 104 may obtain toxicological indicator 108 by receiving a medical input. As used in this disclosure a “medical input” is an element of datum that is obtained relating to the individual's health status. As a non-limiting example, medical input may include a questionnaire and/or survey that identifies a feeling of pain, headache, fever, lethargy, loss of appetite, tenderness, malaise, redness, muscle weakness, and the like thereof. Medical input may include data from an informed advisor as a function of a medical assessment, wherein a “medical assessment” is an evaluation and/or estimation of the individual's health status. As used in this disclosure “informed advisor” is an individual that is skilled in the health and wellness field. As a non-limiting example an informed advisor may include a medical professional who may assist and/or participate in the medical treatment of an individual's health status including, but not limited to, toxicologists, environmental physician, family physicians, endocrinologists, gastroenterologists, internists, oncologists, pediatricians, cardiologists, geneticists, neurologists, physical therapists, primary care providers, and the like thereof. As a non-limiting example, medical input may include an informed advisor that enters a medical assessment comprising a physical exam, neurologic exam, blood test, urine test, imaging test, cellular and/or chemical analysis, genetic test, measurement, visual examination, and the like thereof. As a further non-limiting example, medical input may include a cognitive assessment. As used in this disclosure a “cognitive assessment” is an evaluation and/or estimation of the cognitive functions of an individual. For example, and without limitation, a cognitive assessment may include one or more assessments of memory, behavior, motor function, emotions, and the like thereof. Cognitive assessment may identify one or more feelings and/or cognitive functions of an individual such as, but not limited to, feeling nervous, on edge, restless, unsettled, stressed, surprised, creative, imaginative, daring, adventurous, high energy, low energy, angry, calm, comfortable, contentment, peace, relaxed, loveable, slow moving, fast moving, irritable, impulsive, dull, obsessing, and the like thereof. Cognitive assessment may additionally or alternatively include any cognitive assessment used as a cognitive assessment as described in U.S. Nonprovisional application Ser. No. 17/128,120, filed on Dec. 29, 2020, and entitled “METHODS AND SYSTEMS FOR NOURISHMENT REFINEMENT USING PSYCHIATRIC MARKERS,” the entirety of which is incorporated herein by reference. In another embodiment, and without limitation, medical input may include one or more inputs from a family member. For example, and without limitation, a brother, sister, mother, father, cousin, aunt, uncle, grandparent, child, friend, and the like thereof may enter to computing device 104 that an individual has exhibited health status modifications.

Still referring to FIG. 1, computing device 104 identifies a toxicological profile 112 as a function of toxicological indicator 108. As used in this disclosure a “toxicological profile” is a profile and/or estimation of an individual's health status as pertaining to effects of exposure to at least a xenobiotic, wherein a xenobiotic is described in detail below. For example, and without limitation, toxicological profile 112 may denote that an individual's health system is exhibiting delayed neurological transmissions due to high concentration of lead. As a further non-limiting example, toxicological profile 112 may denote that an individual's health system is vomiting uncontrollably due to high concentrations of mercury. As a further non-limiting example, toxicological profile 112 may denote that an individual's health system is unable to produce ATP as a function of radiation sickness. Computing device 104 identifies toxicological profile 112 as a function of determining at least a xenobiotic 116 as a function of toxicological indicator 108. As used in this disclosure a “xenobiotic” is a substance found within an organism that is not naturally produced or expected to be present within the organism. For example, and without limitation, xenobiotic 116 may include one or more unexpected exposures to a wavelength of light, radioactive doses as measured without limitation in rads, electromagnetic waves, chemicals, infectious agents, heavy metals, inorganic substances, organic substances, and the like thereof. In an embodiment, xenobiotic 116 may include excessive concentrations and/or quantities of zinc, iron, tetrodotoxin, polychlorinated biphenyls, polycyclic aromatic hydrocarbons, trichloroethylene, fentanyl, bisphenol A, triclosan, dimethoate, polyethylene terephthalate, and the like thereof. In another embodiment, xenobiotic 116 may include excessive concentrations and/or quantities of anthrax toxin, botulinum toxin A, tetanus toxin A, diphtheria toxin, dioxin, muscarine, bufotoxin, sarin, hemotoxin, phototoxin, necrotoxin, neurotoxin, and the like thereof.

In an embodiment, and still referring to FIG. 1, determining xenobiotic 116 may include identifying a toxic range. As used in this disclosure a “toxic range” is a range of concentrations associated with the xenobiotic that produce a toxic response in the individual's body. For example, and without limitation, toxic range may denote that a range of 160 mEg/L-200 mEq/L may denote in a toxic effect of sodium in the individual's body. Toxic range may include one or more groupings of toxicity such as, but not limited to, healthy, elevated, dangerous, carcinogenic, disabling, and/or lethal. For example, and without limitation, an elevated toxic range may include a range of 1.10-1.30 mcg/mL, wherein a healthy range is 0.66-1.09 mcg/mL of zinc. As a further non-limiting example, toxic range may include a range and/or limit that denotes a lethal range. For example, and without limitation a limit of a lethal range may denote that any radioactive concentration greater than 1,000 rad will be lethal towards an individual. As a further non-limiting example a healthy range of tetrodotoxin may include 0-5 mcg/kg, wherein an elevated range may include 5.1-25 mcg/kg, wherein a dangerous range may include 25.1-125 mcg/kg, wherein a carcinogenic range may include 125.1-250 mcg/mL, wherein a disabling range may include 250-500 mcg/mL, and wherein a lethal range may include any concentration greater than 500 mcg/kg.

Still referring to FIG. 1, computing device 104 identifies toxicological profile as a function of obtaining an exposure input 120. As used in this disclosure an “exposure input” is an element of datum representing the magnitude and/or length of exposure to a xenobiotic. For example, and without limitation, exposure input 120 may include data denoting that an individual was exposed to ultraviolet A and ultraviolet B waves of light for a period of time, wherein a period of time denotes a measurable value of time such as seconds, minutes, hours, days, weeks, months, years, and the like thereof. As a further non-limiting example, exposure input 120 may include data denoting that an individual consumed 1 kg of arsenic. Exposure input 120 may be determined as a function of one or more questionnaires, surveys, and/or list of questions. In an embodiment, and without limitation, exposure input 120 may be obtained as a function of one or more inputs from an informed advisor, family member, friend, colleague, third party, and the like thereof. In yet another embodiment, exposure input 120 may include input from one or more video monitoring devices, such as but not limited to cameras, camcorders, digital video tape recorders, optical storage mediums, digital encoding devices, and the like thereof. In yet another embodiment, exposure input 120 may be obtained as a function of one or more email records, text messages, and/or other forms of recorded communication. In yet another embodiment, exposure input 108$ may be obtained as a function of a geolocation element. As used in this disclosure a “geolocation element” is an element of data that represents a location position of an individual at a given point in time. For example, and without limitation, geolocation element may include a location within a continent, country, state, city, and the like thereof. Geolocation element may be obtained as a function of one or more wearable devices, wherein a “wearable device”, as used herein, is a device that an individual may wear and/or keep in close proximity that collects, stores, and/or transmits data associated with the individual. For example, and without limitation, geolocation element may be obtained as a function of one or more smartphones, smartwatches, tablets, computers, and the like thereof.

In an embodiment, and still referring to FIG. 1, computing device may obtain exposure input 120 as a function of receiving an exposure route. As used in this disclosure an “exposure route” is a route and/or pathway by which a xenobiotic entered an individual's body. In an embodiment exposure route may include an exposure as a function of a metabolic pathway. For example, and without limitation, metabolic pathway may include one or more pathways such as pentose phosphate pathway, fatty acid synthesis, fatty acid elongation, beta-oxidation, peroxisomal beta-oxidation, glyoxylate cycle, citric acid cycle, urea cycle, and the like thereof. For example, and without limitation, exposure route may include a first compound being ingested into the body, wherein a xenobiotic is produced as a function of a biotransformation reaction due to the citric acid cycle metabolic pathway. For example, and without limitation, exposure route may include one or more pathways such as inhalation, ingestion, and/or direct contact. As a non-limiting example, exposure route may include one or more exposures to groundwater and/or surface water. As a further non-limiting example, exposure route may include one or more exposures to soil, sediment, dust, and the like thereof. As a further non-limiting example, exposure route may include one or more exposures to air. As a further non-limiting example, exposure route may include one or more exposures to food. In an embodiment, exposure route may include one or more routes of entry. For example, a first exposure route may include direct contact with soil as a function of grabbing the soil with a hand, wherein a second exposure route may include ingestion of the soil as a function of not washing the soil from the contaminated location of the hand.

Still referring to FIG. 1, computing device 104 identifies toxicological profile 112 as a function of xenobiotic 116 and exposure input 120 using a profile machine-learning model 124. As used in this disclosure “profile machine-learning model” is a machine-learning model to produce a toxicological profile output given exposure inputs and toxicological indicators as inputs; this is in contrast to a non-machine learning software program where the commands to be executed are determined in advance by a user and written in a programming language. Profile machine-learning model 124 may include one or more profile machine-learning processes such as supervised, unsupervised, or reinforcement machine-learning processes that computing device 104 and/or a remote device may or may not use in the determination of toxicological profile 112. As used in this disclosure “remote device” is an external device to computing device 104. Profile machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train profile machine-learning process as a function of a profile training set. As used in this disclosure “profile training set” is a training set that correlates an exposure input and/or toxicological indicator to a toxicological profile. For example, and without limitation, an exposure input of a 30 minutes of exposure to uranium and a toxicological indicator of a mutation of the BRCA 1 gene may relate to a toxicological profile of increased cellular masses in an individual's breast tissue. The profile training set may be received as a function of user-entered valuations of exposure inputs, toxicological indicators, and/or toxicological profiles. Computing device 104 may receive profile training set by receiving correlations of exposure inputs, and/or toxicological indicators that were previously received and/or determined during a previous iteration of determining toxicological profiles. The profile training set may be received by one or more remote devices that at least correlate an exposure input and/or toxicological indicator to a toxicological profile. The profile training set may be received in the form of one or more user-entered correlations of an exposure input and/or toxicological indicator to a toxicological profile. Additionally or alternatively, a user may include an informed advisor, wherein an informed advisor may include, without limitation, toxicologists, environmental physician, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive profile machine-learning model 124 from a remote device that utilizes one or more profile machine learning processes, wherein a remote device is described above in detail. For example, and without limitation, a remote device may include a computing device, external device, processor, and the like thereof. Remote device may perform the profile machine-learning process using the profile training set to generate toxicological profile 112 and transmit the output to computing device 104. Remote device may transmit a signal, bit, datum, or parameter to computing device 104 that at least relates to toxicological profile 112. Additionally or alternatively, the remote device may provide an updated machine-learning model. For example, and without limitation, an updated machine-learning model may be comprised of a firmware update, a software update, a profile machine-learning process correction, and the like thereof. As a non-limiting example a software update may incorporate a new exposure input that relates to a modified toxicological indicator. Additionally or alternatively, the updated machine learning model may be transmitted to the remote device, wherein the remote device may replace the profile machine-learning model with the updated machine-learning model and determine the toxicological profile as a function of the exposure input using the updated machine-learning model. The updated machine-learning model may be transmitted by the remote device and received by computing device 104 as a software update, firmware update, or corrected profile machine-learning model. For example, and without limitation profile machine-learning model 124 may utilize a random forest machine-learning process, wherein the updated machine-learning model may incorporate a gradient boosting machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

Still referring to FIG. 1, computing device 104 may produce toxicological profile 112 as a function of a classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a processes whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)+P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least one value. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

In an embodiment, and still referring to FIG. 1, computing device 104 may identify toxicological profile 112 as a function of determining a probabilistic vector. As used in this disclosure a “probabilistic vector” is an element of data that represents one or more a quantitative values and/or measures probability associated with a health system modification. For example, and without limitation, probabilistic vector may indicate that a health system has a high probability of developing ischemic heart disease as a function of a large consumption of trans-fats. As a further non-limiting example, probabilistic vector may indicate that an individual's health system has a high likelihood of developing Alzheimer's disease as a function of direct contact with lead-infused paints. As a further non-limiting example, probabilistic vector may indicate that a health system has a low probability of developing cancer as a function of an exposure to uranium for 2 seconds, wherein probabilistic vector may indicate that the health system has a high probability of developing cancer as a function of an exposure to uranium for 20 days.

In an embodiment, and still referring to FIG. 1, probabilistic vector may be determined as a function of a probabilistic machine-learning model. As used in this disclosure a “probabilistic machine-learning model” is a machine-learning model to produce a probabilistic vector output given toxicological indicators as inputs; this is in contrast to a non-machine learning software program where the commands to be executed are determined in advance by a user and written in a programming language. Probabilistic machine-learning model may include one or more probabilistic machine-learning processes such as supervised, unsupervised, or reinforcement machine-learning processes that computing device 104 and/or a remote device may or may not use in the determination of probabilistic vector, wherein a remote device is an external device to computing device 104 as described above in detail. A probabilistic machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train probabilistic machine-learning process as a function of a probabilistic training set. As used in this disclosure a “probabilistic training set” is a training set that correlates at least a toxicological indicator to a probabilistic vector. For example, and without limitation, a toxicological indicator of 20 mg of lead in the circulatory system may relate to a probabilistic vector of 73 for the probability of developing a lead poisoning. The probabilistic training set may be received as a function of user-entered valuations of toxicological indicators, and/or probabilistic vectors. Computing device 104 may receive probabilistic training set by receiving correlations of toxicological indicators and/or probabilistic vectors that were previously received and/or determined during a previous iteration of determining probabilistic vectors. The probabilistic training set may be received by one or more remote devices that at least correlate a toxicological indicator to a probabilistic vector, wherein a remote device is an external device to computing device 104, as described above. Probabilistic training set may be received in the form of one or more user-entered correlations of a toxicological indicator to a probabilistic vector. Additionally or alternatively, a user may include an informed advisor, wherein an informed advisor may include, without limitation, toxicologists, environmental physician, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive probabilistic machine-learning model from a remote device that utilizes one or more probabilistic machine learning processes, wherein remote device is described above in detail. For example, and without limitation, remote device may include a computing device, external device, processor, and the like thereof. Remote device may perform the probabilistic machine-learning process using the probabilistic training set to generate probabilistic vector and transmit the output to computing device 104. Remote device may transmit a signal, bit, datum, or parameter to computing device 104 that at least relates to probabilistic vector. Additionally or alternatively, the remote device may provide an updated machine-learning model. For example, and without limitation, an updated machine-learning model may be comprised of a firmware update, a software update, a probabilistic machine-learning process correction, and the like thereof. As a non-limiting example a software update may incorporate a probabilistic vector that relates to a modified toxicological indicator. Additionally or alternatively, the updated machine learning model may be transmitted to the remote device, wherein the remote device may replace the probabilistic machine-learning model with the updated machine-learning model and determine the probabilistic vector as a function of the toxicological indicator using the updated machine-learning model. The updated machine-learning model may be transmitted by the remote device and received by computing device 104 as a software update, firmware update, or corrected probabilistic machine-learning model. For example, and without limitation a probabilistic machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, probabilistic machine-learning model may identify probabilistic vector as a function of one or more classifiers, wherein a classifier is described above in detail.

Still referring to FIG. 1, computing device 104 may identify toxicological profile 112 as a function of determining a physiological impact. As used in this disclosure a “physiological impact” is an effect that a xenobiotic has on the health system of an individual. For example, and without limitation, physiological impact may include one or more psychological symptoms including, but not limited to, itching, swelling, running nose, watery eyes, coughing, wheezing, hives, rashes, mucus production, anaphylaxis, death, anabolism modifications, catabolism modifications, catecholamine secretion, blood pressure modification, heart rate changes, breathing rate modifications, cravings, constipation, diarrhea, trembling, seizures, sweats, changes in appetite, limb damage, respiratory distress, sleeplessness, and the like thereof. Computing device may determine physiological impact as a function of receiving a binding element. As used in this disclosure a “binding element” is an element of data denoting a target receptor, cell, tissue, and/or organ that xenobiotic 116 binds to. For example, and without limitation, binding element may denote that polycyclic aromatic hydrocarbons bind to the amino group of guanine with 5-nucleophilic sites to prevent DNA replication. As a further non-limiting example, binding element may denote that alpha-latrotoxin binds to the neurexin and/or latrophilin receptors of a cell membrane to induce muscle contractions. In an embodiment binding element from a medical guideline. As used in this disclosure a “medical guideline” is a medical resource that identifies and/or outlines one or more processes in the human body. For example, and without limitation, medical guideline may include medical sources, such as medical textbooks, medical societies, medical organizations, medical websites, and the like thereof. For example, and without limitation, binding element may be received as a function of a medical textbook such as Xenobiotic Metabolism and Disposition. As a further non-limiting example, binding element may be received as a function of a medical website such as WebMD.com, and/or MayoClinic.org.

Still referring to FIG. 1, Physiological impact may be determined as a function of binding element and toxicological indicator 108 using a physiological machine-learning model. As used in this disclosure a “physiological machine-learning model” is a machine-learning model to produce a physiological impact output given binding elements and toxicological indicators as inputs; this is in contrast to a non-machine learning software program where the commands to be executed are determined in advance by a user and written in a programming language. Physiological machine-learning model may include one or more physiological machine-learning processes such as supervised, unsupervised, or reinforcement machine-learning processes that computing device 104 and/or a remote device may or may not use in the determination of physiological impact, wherein a remote device is an external device to computing device 104 as described above in detail. A physiological machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train physiological machine-learning process as a function of a physiological training set. As used in this disclosure a “physiological training set” is a training set that correlates at least a binding element and a toxicological indicator to a physiological impact. For example, and without limitation, binding element of cannabinoid receptors and a toxicological indicator of a 11-OH-tetrahydrocannabinol may relate to a physiological impact of reduced cognitive functioning. The physiological training set may be received as a function of user-entered valuations of binding elements, toxicological indicators, and/or physiological impacts. Computing device 104 may receive physiological training set by receiving correlations of binding elements and/or toxicological indicators that were previously received and/or determined during a previous iteration of determining physiological impacts. The physiological training set may be received by one or more remote devices that at least correlate a binding element and toxicological indicator to a physiological impact, wherein a remote device is an external device to computing device 104, as described above. Physiological training set may be received in the form of one or more user-entered correlations of a binding element and/or toxicological indicator to a physiological impact. Additionally or alternatively, a user may include an informed advisor, wherein an informed advisor may include, without limitation, toxicologists, environmental physician, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive physiological machine-learning model from a remote device that utilizes one or more physiological machine learning processes, wherein remote device is described above in detail. For example, and without limitation, remote device may include a computing device, external device, processor, and the like thereof. Remote device may perform the physiological machine-learning process using the physiological training set to generate physiological impact and transmit the output to computing device 104. Remote device may transmit a signal, bit, datum, or parameter to computing device 104 that at least relates to physiological impact. Additionally or alternatively, the remote device may provide an updated machine-learning model. For example, and without limitation, an updated machine-learning model may be comprised of a firmware update, a software update, a physiological machine-learning process correction, and the like thereof. As a non-limiting example a software update may incorporate a new binding element that relates to a modified toxicological indicator. Additionally or alternatively, the updated machine learning model may be transmitted to the remote device, wherein the remote device may replace the physiological machine-learning model with the updated machine-learning model and determine the physiological as a function of the toxicological indicator using the updated machine-learning model. The updated machine-learning model may be transmitted by the remote device and received by computing device 104 as a software update, firmware update, or corrected physiological machine-learning model. For example, and without limitation a physiological machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference. In an embodiment, and without limitation, physiological machine-learning model may identify physiological impact as a function of one or more classifiers, wherein a classifier is described above in detail.

Still referring to FIG. 1, computing device 104 may identify toxicological profile 112 as a function of receiving a progression element. As used in this disclosure a “progression element” is an element of datum that denotes the progression of a xenobiotic in an individual's body. For example, and without limitation progression element may denote that a xenobiotic may enter an individual's body through inhalation on a first day, wherein the xenobiotic will exit the body one week later from the day of entry into the individual's body. As a further non-limiting example, progression element may denote that a xenobiotic may progress through a series of physiological impacts such as a first impact of a cough, a second impact of a fever, and/or a third impact of nausea prior to being eliminated from the individual's system. Computing device 104 may determine a toxicity stage as a function of the progression element and toxicological indicator. As used in this disclosure a “toxicity stage” is a stage and/or step the individual is experiencing associated with the xenobiotic. For example, and without limitation a toxicity step may denote that an individual is currently in the coughing stage associated with the xenobiotic COVID-19, wherein the next stage is fever and/or pneumonia. As a further non-limiting example, toxicity step may denote than an individual has eliminated 50% of the xenobiotic, wherein the symptoms will start to subside. Computing device 104 may identify toxicological profile 112 as a function of toxicity stage. For example, and without limitation, computing device 104 may identify that an individual's circulatory system may experience reduced blood pressure impacts as a function of the individual being at the beginning stages of anthrax toxin exposure.

Still referring to FIG. 1, computing device 104 may produce toxicological profile 112 by identifying a toxicological ailment. As used in this disclosure a “toxicological ailment” is an ailment and/or collection of ailments that impact an individual's health status. As a non-limiting example toxicological ailment may include asthma, birth defects, dermatitis, emphysema, fertility problems, heart disease, immune deficiency diseases, job-related illness, lead poisoning, mercury poisoning, pneumoconiosis, Queensland fever, sunburn, tooth decay, uranium poisoning, vision disorder, xeroderma pigmentosa, yusho poisoning, zinc deficiency, zinc poisoning, type II diabetes, metabolic syndrome, myocardial infarction, hypertension, coronary heart disease, autism spectrum disorder, attention deficit hyperactivity disorder, Alzheimer's disease, Parkinson's disease, amyotrophic lateral sclerosis, neurological impairments, systemic sensitization autoimmune diseases, asthma, chronic obstructive pulmonary diseases, childhood leukemia, childhood cancers, breast cancer, prostate cancer, kidney cancer, and the like thereof. Toxicological ailment may be identified as a function of one or more ailment machine-learning models. As used in this disclosure “ailment machine-learning model” is a machine-learning model to produce a toxicological ailment output given toxicological indicators as inputs; this is in contrast to a non-machine learning software program where the commands to be executed are determined in advance by a user and written in a programming language. Ailment machine-learning model may include one or more ailment machine-learning processes such as supervised, unsupervised, or reinforcement machine-learning processes that computing device 104 and/or a remote device may or may not use in the determination of toxicological ailment. An ailment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train ailment machine-learning process as a function of an ailment training set. As used in this disclosure “ailment training set” is a training set that correlates a toxicological indicator to a toxicological ailment. As a non-limiting example a toxicological indicator of a lump in breast tissue may relate to a toxicological ailment of breast cancer. The ailment training set may be received as a function of user-entered valuations of toxicological indicators and/or toxicological ailments. Computing device 104 may receive ailment training by receiving correlations of toxicological indicators and/or toxicological ailments that were previously received and/or determined during a previous iteration. The ailment training set may be received by one or more remote devices that at least correlate toxicological indicators to toxicological ailments, wherein a remote device is an external device to computing device 104, as described above. The ailment training set may be received by one or more user-entered correlations of toxicological indicators to toxicological ailments. Additionally or alternatively, a user may include an informed advisor, wherein an informed advisor may include, without limitation, toxicologists, environmental physician, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive ailment machine-learning model from a remote device that utilizes one or more ailment machine learning processes, wherein a remote device is described above in detail. For example, and without limitation, a remote device may include a computing device, external device, processor, and the like thereof. Remote device may perform the ailment machine-learning process using the ailment training set to generate toxicological ailment and transmit the output to computing device 104. Remote device may transmit a signal, bit, datum, or parameter to computing device 104 that at least relates to toxicological ailments. Additionally or alternatively, the remote device may provide an updated machine-learning model. For example, and without limitation, an updated machine-learning model may be comprised of a firmware update, a software update, an ailment machine-learning process correction, and the like thereof. As a non-limiting example a software update may incorporate a new toxicological indicator that relates to a modified toxicological ailment. Additionally or alternatively, the updated machine learning model may be transmitted to the remote device, wherein the remote device may replace the ailment machine-learning model with the updated machine-learning model and determine the toxicological ailment as a function of the toxicological indicator using the updated machine-learning model. The updated machine-learning model may be transmitted by the remote device and received by computing device 104 as a software update, firmware update, or corrected ailment machine-learning model. For example, and without limitation ailment machine-learning model may utilize a neural net machine-learning process, wherein the updated machine-learning model may incorporate hierarchical clustering machine-learning process.

Still referring to FIG. 1, computing device 104 determines an edible 128 as a function of toxicological profile 112. As used in this disclosure an “edible” is a source of nourishment that may be consumed by a user such that the user may absorb the nutrients from the source. For example and without limitation, an edible may include legumes, plants, fungi, nuts, seeds, breads, dairy, eggs, meat, cereals, rice, seafood, desserts, dried foods, dumplings, pies, noodles, salads, stews, soups, sauces, sandwiches, and the like thereof. In an embodiment, edible may be determined as a function of a physiological response, wherein a “physiological response” is a reaction and/or response to an edible. For example, and without limitation, a first edible may be identified for a first physiological response and a second edible may be determined for a second physiological response. For example, and without limitation, a first edible of steak may be determined for a first physiological response of radiation, wherein a second edible of ginger may identified for a second physiological response of an allergic reaction. Computing device 104 may determine edible 128 as a function of receiving a nourishment composition. As used in this disclosure a “nourishment composition” is a list and/or compilation of all of the nutrients contained in an edible. As a non-limiting example nourishment composition may include one or more quantities and/or amounts of total fat, including saturated fat and/or trans-fat, cholesterol, sodium, total carbohydrates, including dietary fiber and/or total sugars, protein, vitamin A, vitamin C, thiamin, riboflavin, niacin, pantothenic acid, vitamin b6, folate, biotin, vitamin B12, vitamin D, vitamin E, vitamin K, calcium, iron, phosphorous, iodine, magnesium, zinc, selenium, copper, manganese, chromium, molybdenum, chloride, and the like thereof.

In an embodiment, and still referring to FIG. 1, nourishment composition may be obtained as a function of an edible directory, wherein an “edible directory” is a database of edibles that may be identified as a function of one or more metabolic components. Edible directory may be implemented, without limitation, as a relational databank, a key-value retrieval databank such as a NOSQL databank, or any other format or structure for use as a databank that a person skilled in the art would recognize as suitable upon review of the entirety of this disclosure. Edible directory may alternatively or additionally be implemented using a distributed data storage protocol and/or data structure, such as a distributed hash table or the like. Edible directory may include a plurality of data entries and/or records as described above. Data entries in a databank may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a databank may store, retrieve, organize, and/or reflect data and/or records as used herein, as well as categories and/or populations of data consistently with this disclosure. Edible directory may include a carbohydrate tableset. Carbohydrate tableset may relate to a nourishment composition of an edible with respect to the quantity and/or type of carbohydrates in the edible. As a non-limiting example, carbohydrate tableset may include monosaccharides, disaccharides, oligosaccharides, polysaccharides, and the like thereof. Edible directory may include a fat tableset. Fat tableset may relate to a nourishment composition of an edible with respect to the quantity and/or type of esterified fatty acids in the edible. Fat tableset may include, without limitation, triglycerides, monoglycerides, diglycerides, phospholipids, sterols, waxes, and free fatty acids. Edible directory may include a fiber tableset. Fiber tableset may relate to a nourishment composition of an edible with respect to the quantity and/or type of fiber in the edible. As a non-limiting example, fiber tableset may include soluble fiber, such as beta-glucans, raw guar gum, psyllium, inulin, and the like thereof as well as insoluble fiber, such as wheat bran, cellulose, lignin, and the like thereof. Edible directory may include a mineral tableset. Mineral tableset may relate to a nourishment composition of an edible with respect to the quantity and/or type of minerals in the edible. As a non-limiting example, mineral tableset may include calcium, phosphorous, magnesium, sodium, potassium, chloride, sulfur, iron, manganese, copper, iodine, zing, cobalt, fluoride, selenium, and the like thereof. Edible directory may include a protein tableset. Protein tableset may relate to a nourishment composition of an edible with respect to the quantity and/or type of proteins in the edible. As a non-limiting example, protein tableset may include amino acids combinations, wherein amino acids may include, without limitation, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and the like thereof. Edible directory may include a vitamin tableset. Vitamin tableset may relate to a nourishment composition of an edible with respect to the quantity and/or type of vitamins in the edible. As a non-limiting example, vitamin tableset may include vitamin A, vitamin B₁, vitamin B₂, vitamin B₃, vitamin B₅, vitamin B₆, vitamin B₇, vitamin B₉, vitamin B₁₂, vitamin C, vitamin D, vitamin E, vitamin K, and the like thereof.

Still referring to FIG. 1, computing device 104 may produce a nourishment desideration as a function of toxicological profile 112. As used in this disclosure a “nourishment desideration” is requirement and/or necessary amount of nutrients required for a user to consume. As a non-limiting example, nourishment desideration may include a user requirement of 35 g of fiber to be consumed per day. Nourishment desideration may be determined as a function of receiving a nourishment goal. As used in this disclosure a “nourishment goal” is a recommended amount of nutrients that a user should consume. Nourishment goal may be identified by one or more organizations that relate to, represent, and/or study toxicological ailments in humans, such as the American Medical Association, American Autoimmune Related Diseases Association, Society of toxicology, society of environmental toxicology, American Academy of Clinical Toxicology, American College of Toxicology, Agency for Toxic Substances, British Toxicology Society, and the like thereof.

Still referring to FIG. 1, computing device 104 identifies edible 128 as a function of nourishment composition, nourishment desideration, and an edible machine-learning model. As used in this disclosure a “edible machine-learning model” is a machine-learning model to produce an edible output given nourishment compositions and nourishment desiderations as inputs; this is in contrast to a non-machine learning software program where the commands to be executed are determined in advance by a user and written in a programming language. Edible machine-learning model may include one or more edible machine-learning processes such as supervised, unsupervised, or reinforcement machine-learning processes that computing device 104 and/or a remote device may or may not use in the determination of edible 128, wherein a remote device is an external device to computing device 104 as described above in detail. An edible machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train edible machine-learning process as a function of an edible training set. As used in this disclosure an “edible training set” is a training set that correlates at least nourishment composition and nourishment desideration to an edible. For example, and without limitation, nourishment composition of 100 mg of vitamin C and a nourishment desideration of 90 mg of vitamin C as a function of childhood leukemia may relate to an edible of oranges. The edible training set may be received as a function of user-entered valuations of nourishment compositions, nourishment desiderations, and/or edibles. Computing device 104 may receive edible training set by receiving correlations of nourishment compositions and/or nourishment desiderations that were previously received and/or determined during a previous iteration of determining edibles. The edible training set may be received by one or more remote devices that at least correlate a nourishment composition and nourishment desideration to an edible, wherein a remote device is an external device to computing device 104, as described above. Edible training set may be received in the form of one or more user-entered correlations of a nourishment composition and/or nourishment desideration to an edible. Additionally or alternatively, a user may include an informed advisor, wherein an informed advisor may include, without limitation toxicologists, environmental physician, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive edible machine-learning model from a remote device that utilizes one or more edible machine learning processes, wherein remote device is described above in detail. For example, and without limitation, remote device may include a computing device, external device, processor, and the like thereof. Remote device may perform the edible machine-learning process using the edible training set to generate edible 128 and transmit the output to computing device 104. Remote device may transmit a signal, bit, datum, or parameter to computing device 104 that at least relates to edible 128. Additionally or alternatively, the remote device may provide an updated machine-learning model. For example, and without limitation, an updated machine-learning model may be comprised of a firmware update, a software update, an edible machine-learning process correction, and the like thereof. As a non-limiting example a software update may incorporate a new nourishment composition that relates to a modified nourishment desideration. Additionally or alternatively, the updated machine learning model may be transmitted to the remote device, wherein the remote device may replace the edible machine-learning model with the updated machine-learning model and determine the edible as a function of the nourishment desideration using the updated machine-learning model. The updated machine-learning model may be transmitted by the remote device and received by computing device 104 as a software update, firmware update, or corrected edible machine-learning model. For example, and without limitation an edible machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference. In an embodiment, and without limitation, edible machine-learning model may identify edible 128 as a function of one or more classifiers, wherein a classifier is described above in detail.

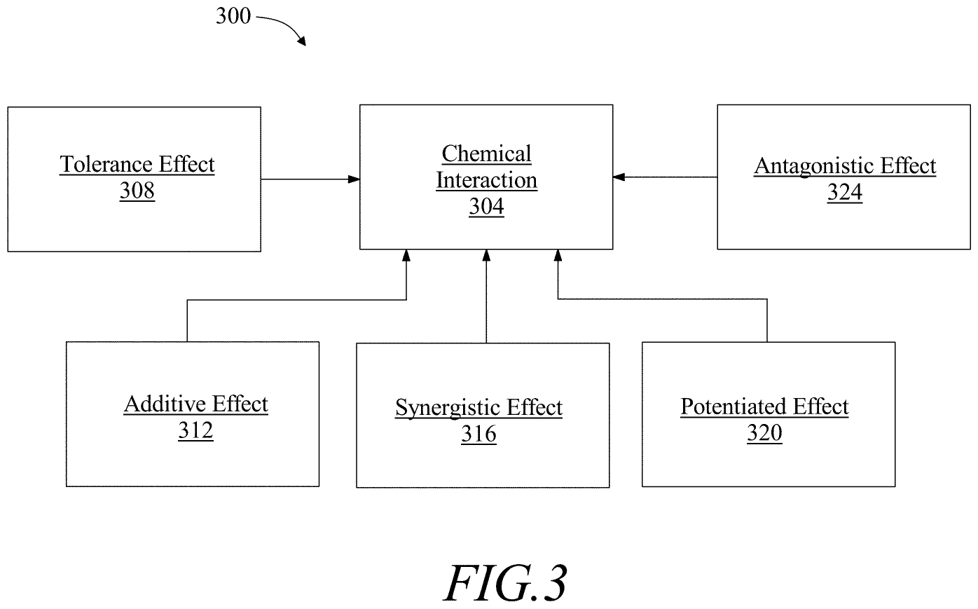

Still referring to FIG. 1, computing device 104 may identify edible as a function of a likelihood parameter. As used in this disclosure a “likelihood parameter” is a parameter that identities the probability of a user to consume an edible. As a non-limiting example likelihood parameter may identify a high probability that a user will consume an edible of chicken. As a further non-limiting example likelihood parameter may identify a low probability that a user will consume an edible of spinach. Likelihood parameter may be determined as a function of a user taste profile. As used in this disclosure a “user taste profile” is a profile of a user that identifies one or more desires, preferences, wishes, and/or wants that a user has. As a non-limiting example a user taste profile may include a user's preference for cinnamon flavor and/or crunchy textured edibles. Likelihood parameter may be determined as a function of an edible profile. As used in this disclosure an “edible profile” is taste of an edible is the sensation of flavor perceived in the mouth and throat on contact with the edible. Edible profile may include one or more flavor variables. As used in this disclosure a “flavor variable” is a variable associated with the distinctive taste of an edible, wherein a distinctive may include, without limitation sweet, bitter, sour, salty, umami, cool, and/or hot. Edible profile may be determined as a function of receiving flavor variable from a flavor directory. As used in this disclosure a “flavor directory” is a database or other data structure including flavors for an edible. As a non-limiting example flavor directory may include a list and/or collection of edibles that all contain sweet flavor variables. As a further non-limiting example flavor directory may include a list and/or collection of edibles that all contain sour flavor variables. Flavor directory may be implemented similarly to an edible directory as described below in detail, in reference to FIG. 3. Likelihood parameter may alternatively or additionally include any user taste profile and/or edible profile used as a likelihood parameter as described in U.S. Nonprovisional application Ser. No. 17/032,080, filed on Sep. 25, 2020, and entitled “METHODS, SYSTEMS, AND DEVICES FOR GENERATING A REFRESHMENT INSTRUCTION SET BASED ON INDIVIDUAL PREFERENCES,” the entirety of which is incorporated herein by reference.

Still referring to FIG. 1, computing device 104 may determine edible 128 as a function of identifying a toxic response vector. As used in this disclosure a “toxic response vector” is a data structure that represents one or more a quantitative values and/or measures toxic responses in an individual's body. A vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes. For example, and without limitation, toxic response vector may denote that a response of a cough due to a xenobiotic of anthrax toxin exposure may develop into respiratory failure. In an embodiment, and without limitation, toxic response vector may include a dose-response curve. As used in this disclosure a “dose-response curve” is graphical representation of the magnitude of the response of an individual's body as a function of the concentration and/or exposure of the xenobiotic. For example, and without limitation, dose-response curve may denote one or more safe and/or unsafe regions of dosages as a function of the expected response.

In an embodiment, and still referring to FIG. 1, toxic response vector may be determined as a function of a response machine-learning model. As used in this disclosure a “response machine-learning model” is a machine-learning model to produce a toxic response vector output given toxicological profiles as inputs; this is in contrast to a non-machine learning software program where the commands to be executed are determined in advance by a user and written in a programming language. Response machine-learning model may include one or more response machine-learning processes such as supervised, unsupervised, or reinforcement machine-learning processes that computing device 104 and/or a remote device may or may not use in the determination of toxic response vector, wherein a remote device is an external device to computing device 104 as described above in detail. A response machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train response machine-learning process as a function of a response training set. As used in this disclosure a “response training set” is a training set that correlates at least a toxicological profile to a toxic response vector. For example, and without limitation, a toxicological profile of a likelihood for developing skin cancer due to overexposure of the UV rays of the sun to a toxic response vector of 78 for developing third degree burns and/or mutations associated with melanoma. The response training set may be received as a function of user-entered valuations of toxicological profiles, and/or toxic response vectors. Computing device 104 may receive response training set by receiving correlations of toxicological profiles and/or toxic response vectors that were previously received and/or determined during a previous iteration of determining toxic response vectors. The response training set may be received by one or more remote devices that at least correlate a toxicological profile to a toxic response vector, wherein a remote device is an external device to computing device 104, as described above. Response training set may be received in the form of one or more user-entered correlations of a toxicological profile to a toxic response vector. Additionally or alternatively, a user may include an informed advisor, wherein an informed advisor may include, without limitation, toxicologists, environmental physician, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive response machine-learning model from a remote device that utilizes one or more response machine learning processes, wherein remote device is described above in detail. For example, and without limitation, remote device may include a computing device, external device, processor, and the like thereof. Remote device may perform the response machine-learning process using the response training set to generate toxic response vector and transmit the output to computing device 104. Remote device may transmit a signal, bit, datum, or parameter to computing device 104 that at least relates to toxic response vector. Additionally or alternatively, the remote device may provide an updated machine-learning model. For example, and without limitation, an updated machine-learning model may be comprised of a firmware update, a software update, a response machine-learning process correction, and the like thereof. As a non-limiting example a software update may incorporate a toxic response vector that relates to a modified toxicological profile. Additionally or alternatively, the updated machine learning model may be transmitted to the remote device, wherein the remote device may replace the response machine-learning model with the updated machine-learning model and determine the toxic response vector as a function of the toxicological profile using the updated machine-learning model. The updated machine-learning model may be transmitted by the remote device and received by computing device 104 as a software update, firmware update, or corrected response machine-learning model. For example, and without limitation a response machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, response machine-learning model may identify toxic response vector as a function of one or more classifiers, wherein a classifier is described above in detail.

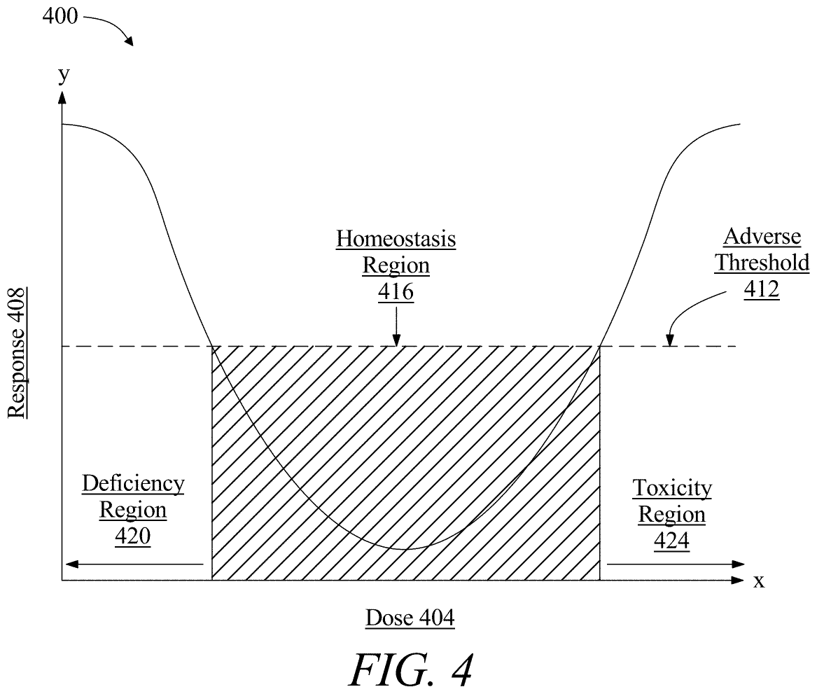

Still referring to FIG. 1, toxic response vector may be identified as a function of determining a hormetic element. As used in this disclosure a “hormetic element” is an element of data denoting that a xenobiotic exhibits hormesis, wherein hormesis denotes a biphasic dose response to a xenobiotic such that a low dose results in a beneficial effect and a high dose and/or no dose results in toxicity and/or deficiency. For example, and without limitation, hormetic element may include one or more regions of deficiency, homeostasis, and/or toxicity as described in detail below, in reference to FIG. 4. Computing device 104 may identify toxic response vector as a function of hormetic element and toxicological profile 112 using a response machine-learning model. As used in this disclosure a “response machine-learning model” is a machine-learning model to produce a toxic response vector output given hormetic elements and toxicological profiles 112 as inputs; this is in contrast to a non-machine learning software program where the commands to be executed are determined in advance by a user and written in a programming language. Response machine-learning model may include one or more response machine-learning processes such as supervised, unsupervised, or reinforcement machine-learning processes that computing device 104 and/or a remote device may or may not use in the determination of edible 128, wherein a remote device is an external device to computing device 104 as described above in detail. A response machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train response machine-learning process as a function of a response training set. As used in this disclosure an “response training set” is a training set that correlates at least a hormetic element and toxicological profile 112 to a toxic response vector. For example, and without limitation, hormetic element toxicity of uranium and a toxicological profile 112 of breast cancer as a function of living near a nuclear power plant may relate to an edible of seafood. The response training set may be received as a function of user-entered valuations of hormetic elements, toxicological profiles 112, and/or toxic response vectors. Computing device 104 may receive response training set by receiving correlations of hormetic elements and/or toxicological profiles 112 that were previously received and/or determined during a previous iteration of determining toxic response vectors. The response training set may be received by one or more remote devices that at least correlate a hormetic element and toxicological profile 112 to a toxic response profile, wherein a remote device is an external device to computing device 104, as described above. Response training set may be received in the form of one or more user-entered correlations of a hormetic element and/or toxicological profile 112 to a toxic response vector. Additionally or alternatively, a user may include an informed advisor, wherein an informed advisor may include, without limitation toxicologists, environmental physician, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive response machine-learning model from a remote device that utilizes one or more response machine learning processes, wherein remote device is described above in detail. For example, and without limitation, remote device may include a computing device, external device, processor, and the like thereof. Remote device may perform the response machine-learning process using the response training set to generate toxic response vector and transmit the output to computing device 104. Remote device may transmit a signal, bit, datum, or parameter to computing device 104 that at least relates to toxic response vector. Additionally or alternatively, the remote device may provide an updated machine-learning model. For example, and without limitation, an updated machine-learning model may be comprised of a firmware update, a software update, a response machine-learning process correction, and the like thereof. As a non-limiting example a software update may incorporate a new hormetic element that relates to a modified toxicological profile 112. Additionally or alternatively, the updated machine learning model may be transmitted to the remote device, wherein the remote device may replace the response machine-learning model with the updated machine-learning model and determine the toxic response vector as a function of the toxicological profile 112 using the updated machine-learning model. The updated machine-learning model may be transmitted by the remote device and received by computing device 104 as a software update, firmware update, or corrected response machine-learning model. For example, and without limitation a response machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, response machine-learning model may identify toxic response vector as a function of one or more classifiers, wherein a classifier is described above in detail.

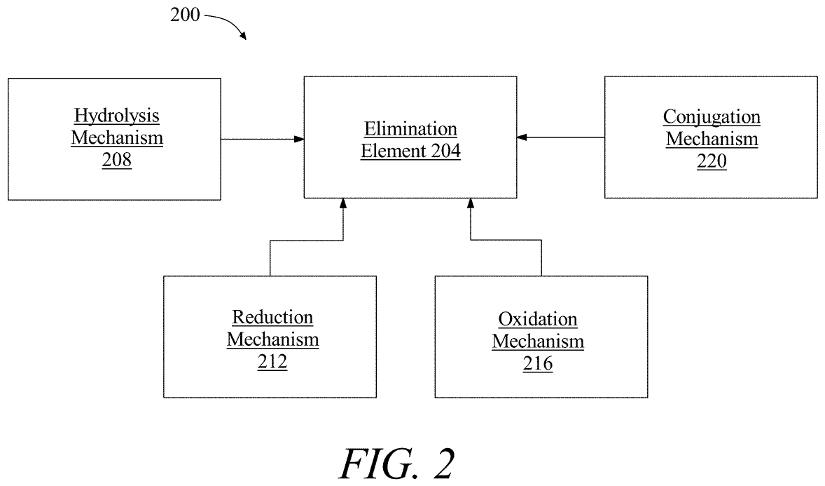

In an embodiment, and still referring to FIG. 1, computing device 104 may determine edible 128 as a function of obtaining an elimination element, wherein an elimination element is an element of data associated with the clearance of the xenobiotic from the individual's body, as described below in detail, in reference to FIG. 2. For example, and without limitation, elimination element may include one or more mechanisms of elimination such as a hydrolysis mechanism, reduction mechanism, oxidation mechanism, conjugation mechanism, and the like thereof. In an embodiment, computing device 104 may determine a first edible as a function of a first xenobiotic comprising a first clearance rate, wherein a second edible may be determined as a function of the first xenobiotic comprising the first clearance rate and a second xenobiotic comprising a second clearance rate. For example, and without limitation, a first edible of salmon may be identified for a first xenobiotic polycyclic aromatic hydrocarbons, wherein a second edible of broccoli may be determined as a function of the first xenobiotic and a second xenobiotic trichloroethylene.

Still referring to FIG. 1, computing device 104 generates a nourishment program 132 as a function of edible 128. As used in this disclosure a “nourishment program” is a program consisting of one or more edibles that are to be consumed over a given time period, wherein a time period is a temporal measurement such as seconds, minutes, hours, days, weeks, months, years, and the like thereof. As a non-limiting example nourishment program 132 may consist of recommending carrots for 8 days. As a further non-limiting example nourishment program 132 may recommend shellfish for a first day, legumes for a second day, and steak for a third day. Nourishment program 132 may include one or more diet programs such as paleo, keto, vegan, vegetarian, Mediterranean, Dukan, Zone, HCG, and the like thereof. Computing device 104 may develop nourishment program 132 as a function of a toxicological functional goal. As used in this disclosure an “toxicological functional goal” is a goal that an edible may generate according to a predicted and/or purposeful plan. As a non-limiting example, toxicological functional goal may include a treatment goal. As used in this disclosure a “treatment goal” is a toxicological functional goal that is designed to at least reverse and/or eliminate toxicological indicator 108, toxicological profile 112, and/or toxicological ailment. As a non-limiting example, a treatment goal may include reversing the effects of the toxicological ailment prostate cancer. As a further non-limiting example, a treatment goal includes reversing the toxicological ailment of rheumatoid arthritis osteoporosis. Toxicological functional goal may include a prevention goal. As used in this disclosure a “prevention goal” is a toxicological functional goal that is designed to at least prevent and/or avert toxicological indicator 108, toxicological profile 112, and/or toxicological ailment. As a non-limiting example, a prevention goal may include preventing the development of the toxicological ailment of chronic obstructive pulmonary disease. Toxicological functional goal may include a mitigation goal. As used in this disclosure a “mitigation goal” is a functional goal that is designed to reduce the symptoms and/or effects of a toxicological ailment. For example, and without limitation, mitigation goal may include reducing the effects of the toxicological ailment autism spectrum disorder. Additionally or alternatively, toxicological functional goal may include one or more goals associated with epigenetic alteration and/or gene therapy to alter a mutation and/or modification of an individual's nuclear code.

Still referring to FIG. 1, computing device 104 may develop nourishment program 132 as a function of edible 128 and toxicological functional goal using a nourishment machine-learning model. As used in this disclosure a “nourishment machine-learning model” is a machine-learning model to produce a nourishment program output given edibles and/or toxicological functional goals as inputs; this is in contrast to a non-machine learning software program where the commands to be executed are determined in advance by a user and written in a programming language. Nourishment machine-learning model may include one or more nourishment machine-learning processes such as supervised, unsupervised, or reinforcement machine-learning processes that computing device 104 and/or a remote device may or may not use in the development of nourishment program 132. Nourishment machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 1, computing device 104 may train nourishment machine-learning process as a function of a nourishment training set. As used in this disclosure a “nourishment training set” is a training set that correlates a toxicological functional goal to an edible. The nourishment training set may be received as a function of user-entered edibles, toxicological functional goals, and/or nourishment programs. For example, and without limitation, a toxicological functional goal of treating type II diabetes may correlate to an edible of salmon. Computing device 104 may receive nourishment training by receiving correlations of toxicological functional goals and/or edibles that were previously received and/or determined during a previous iteration of developing nourishment programs. The nourishment training set may be received by one or more remote devices that at least correlate a toxicological functional goal and/or edible to a nourishment program, wherein a remote device is an external device to computing device 104, as described above. Nourishment training set may be received in the form of one or more user-entered correlations of a toxicological functional goal and/or edible to a nourishment program. Additionally or alternatively, a user may include an informed advisor, wherein an informed advisor may include, without limitation toxicologists, environmental physician, family physicians, endocrinologists, gastroenterologists, internists, oncologists, pediatricians, cardiologists, geneticists, neurologists, physical therapists, primary care providers, and the like thereof.

Still referring to FIG. 1, computing device 104 may receive nourishment machine-learning model from the remote device that utilizes one or more nourishment machine learning processes, wherein a remote device is described above in detail. For example, and without limitation, a remote device may include a computing device, external device, processor, and the like thereof. The remote device may perform the nourishment machine-learning process using the nourishment training set to develop nourishment program 132 and transmit the output to computing device 104. The remote device may transmit a signal, bit, datum, or parameter to computing device 104 that at least relates to nourishment program 132. Additionally or alternatively, the remote device may provide an updated machine-learning model. For example, and without limitation, an updated machine-learning model may be comprised of a firmware update, a software update, a nourishment machine-learning process correction, and the like thereof. As a non-limiting example a software update may incorporate a new toxicological functional goal that relates to a modified edible. Additionally or alternatively, the updated machine learning model may be transmitted to the remote device, wherein the remote device may replace the nourishment machine-learning model with the updated machine-learning model and develop the nourishment program as a function of the toxicological functional goal using the updated machine-learning model. The updated machine-learning model may be transmitted by the remote device and received by computing device 104 as a software update, firmware update, or corrected nourishment machine-learning model. For example, and without limitation nourishment machine-learning model may utilize a neural net machine-learning process, wherein the updated machine-learning model may incorporate decision tree machine-learning processes.

Now referring to FIG. 2, an exemplary embodiment 200 of an elimination element 204. As used in this disclosure an “elimination element” is an element of data associated with the clearance of the xenobiotic from the individual's body. In an embodiment, and without limitation, elimination element 204 may denote a biotransformation mechanism, clearance rate, half-life, chelation mechanism, and/or elimination rate of a xenobiotic. For example, and without limitation, elimination element may denote that a half-life for the xenobiotic PCB-170 is 15.5 years. As a further non-limiting example, elimination element 204 may denote that a porphyrin chelator may be utilized to eliminate excessive iron from the human body. Elimination element 204 may identify a hydrolysis mechanism 208. As used in this disclosure a “hydrolysis mechanism” is a biotransformation process that incorporates water in the chemical reaction. Hydrolysis mechanism 208 may include one or more molecules capable of performing hydrolysis such as, but not limited to, carboxylesterases, alkaline phosphatases, peptidases, epoxide hydrolases, and the like thereof. Elimination element 204 may identify a reduction mechanism 212. As used in this disclosure a “reduction mechanism” is a biotransformation process that adds electrons to the xenobiotic. For example, and without limitation, xenobiotics containing compounds such as aldehydes, ketones, disulfides, quinones, alkenes, azos, nitros, and the like thereof may be reduced to aid in elimination. In an embodiment, reduction mechanism 212 may include one or more molecules capable of reducing a xenobiotic such as, but not limited to, flavoproteins, alcohol dehydrogenase, aldehyde oxidase, cytochrome P450, and the like thereof. Elimination element 204 may include an oxidation mechanism 216. As used in this disclosure an “oxidation mechanism” is a biotransformation process that removes electrons from the xenobiotic. For example, and without limitation, oxidation mechanism 216 may include a process that catalyzes monooxygenation of heme containing proteins. As a further non-limiting example, oxidation mechanism 216 may include one or more molecules capable of oxidizing xenobiotics such as, but not limited to cytochrome P450, alcohol dehydrogenase, aldehyde dehydrogenase, dihydrodiol dehydrogenase, molybdenum hydroxylase, xanthine oxidoreductase, aldehyde oxidase, monoamine oxidase, peroxidase-dependent co-oxidation, flavin monooxygenase, and the like thereof. Elimination element 204 may include a conjugation mechanism 220. As used in this disclosure a “conjugation mechanism” is a biotransformation process that attaches a small functional group to a xenobiotic. For example, and without limitation, a small functional group may include a primary alcohol, secondary alcohol, phenol, catechol, N-oxide, aliphatic amine, aromatic amine, aromatic hydroxylamine, aromatic hydroxyamide, and the like thereof. In an embodiment conjugation mechanism 220 may include one or more transferase enzymes, such as but not limited to a glucuronidation enzyme, sulfonation enzyme, acetylation enzyme, methylation enzyme, glutathione enzyme, amino acid enzyme, and the like thereof.

Now referring to FIG. 3, an exemplary embodiment 300 of a chemical interaction 304 is discussed. As used in this disclosure a “chemical interaction” is an interaction of two or more chemicals, molecules, and/or substances that occurs within an individual's body. For example, a chemical interaction may include an absorption, biotransformation, protein binding, activation, deactivation, and the like thereof. Chemical interaction 304 may include a tolerance effect 308. As used in this disclosure a “tolerance effect” is an impact on the individual's body such that a state of decreased responsiveness to the chemical occurs. For example, and without limitation tolerance effect 308 may include a downregulation of receptors, a decreased secretion of chemicals, reduced chemical transport mechanisms, and the like thereof. Chemical interaction 304 may include an additive effect 312. As used in this disclosure an “additive effect” is an event that occurs that impacts an individual's body, wherein the impact constitutes a combined effect of the 2 or more chemicals. For example, and without limitation, additive effect 312 may include a sum of effects and/or impacts of each chemical. Chemical interaction 304 may include a synergistic effect 316. As used in this disclosure a “synergistic effect” is an event that occurs that impacts an individual's body, wherein the impact is greater than the sum of the effect of each chemical alone. For example, and without limitation, synergistic effect 316 may denote that a first chemical should respond with coughing and a second chemical should response with sneezing, wherein the actual response of the first and second chemical combined is anaphylaxis. Chemical interaction 304 may include a potentiated effect 320. As used in this disclosure a “potentiated effect” is an impact on an individual's body as a function of a first chemical becoming more toxic due to a second chemical. For example, and without limitation, isopropanol, which is not hepatotoxic, may interact with carbon tetrachloride, wherein the hepatotoxicity of the carbon tetrachloride may increase due to the interaction with the isopropanol. Chemical interaction 304 may include an antagonistic effect 324. As used in this disclosure an “antagonistic effect” is an impact on the individual's body that is reduced and/or mitigated as a function of a first chemical interfering with the effects of a second chemicals. For example, and without limitation, antagonistic effect 324 may include a first chemical that reduces blood pressure, wherein a second chemical increase blood pressure such that the first chemical effect is mitigated and/or eliminated. Antagonistic effect 324 may include, without limitation, functional antagonism, chemical antagonism, dispositional antagonism, receptor antagonism, and the like thereof.

Now referring to FIG. 4, an exemplary embodiment 400 of a hormetic element is illustrated. Hormetic element may include a dose 404. As used in this disclosure a “dose” is a measurable value representing the quantity of the xenobiotic that is interacting with the individual. For example, and without limitation, dose 404 may include one or more weights of a xenobiotic, volume of a xenobiotic solution, number of xenobiotic dosage forms, and the like thereof. For example, and without limitation, dose 404 may include quantities including pg, mcg, mg, g, kg, pL, mcL, mL, L, IU, and the like thereof. Hormetic element may include a response 408. As used in this disclosure a “response” is a reaction and/or process the individual's body performs as a function of interacting with the xenobiotic. For example, and without limitation, response 408 may include itchiness, swelling, running nose, watery eyes, coughing, wheezing, hives, rashes, mucus production, anaphylaxis, death, anabolism, catabolism, catecholamine secretion, blood pressure modification, heart rate changes, breathing rate modifications, and the like thereof. In an embodiment, an adverse threshold 412 may be determined as a function of response 408. As used in this disclosure an “adverse threshold” is a limit that a response should never exceed. For example, and without limitation, adverse threshold 412 may denote that a response should not include anaphylaxis, difficulty breathing, and/or increased blood pressure. Hormetic element may include a homeostasis region 412. As used in this disclosure a “homeostasis region” is a region representing the required dose quantities and response to perform homeostatic processes. For example, and without limitation, a normal dose of zinc may include 0.66-1.10 mcg/mL of zinc, wherein a normal response is to transport oxygen throughout the circulatory system. Hormetic element may include a deficiency region 420. As used in this disclosure a “deficiency region” is a region representing that a required xenobiotic is inadequate to perform necessary homeostatic processes such that a response above the adverse threshold occurs. For example, and without limitation, deficiency region may denote that to perform the homeostatic process of calcium absorption, vitamin D is required to be greater than and/or equal to 20 ng/mL. Hormetic element may include a toxicity region 424. As used in this disclosure a “toxicity region” is a region representing that a required xenobiotic is more concentrated than allowable to perform necessary homeostatic processes such that a response above the adverse threshold occurs. For example, and without limitation, toxicity region may denote that ethyl alcohol in concentrations that are greater than and/or equal to 0.4% of blood alcohol content disrupts the normal homeostatic process of cognitive function such that a response of incapacitation occurs.

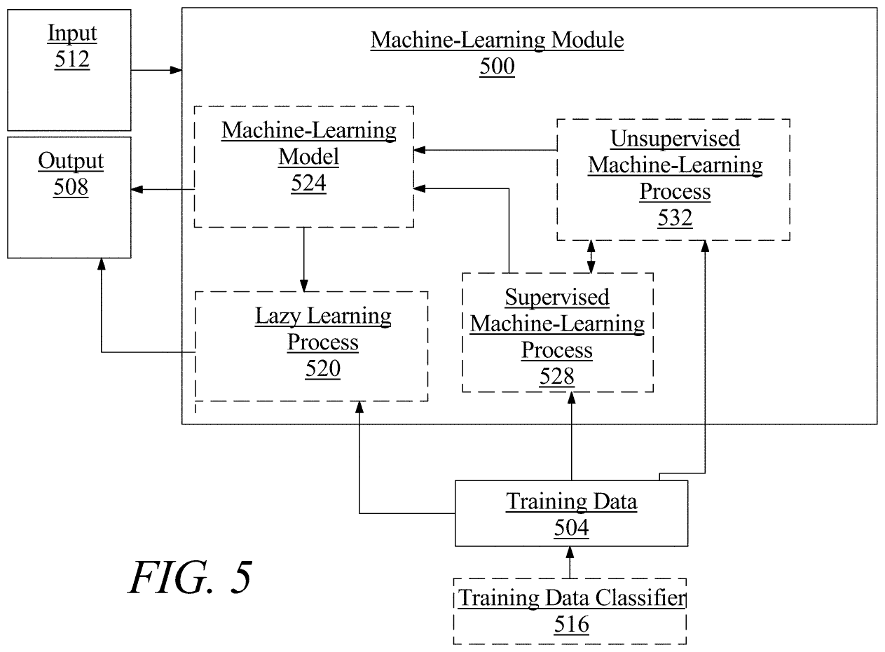

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example inputs of xenobiotics and/or exposure inputs may output toxicological profiles.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to sub-categories of xenobiotics such as toxins and/or toxic agents.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include xenobiotics and/or exposure inputs as described above as inputs, toxicological profiles as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

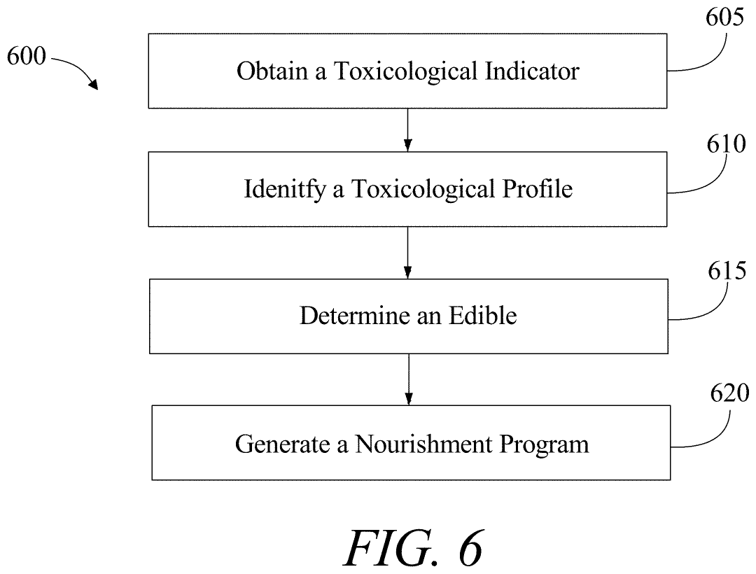

Now referring to FIG. 6, an exemplary embodiment 600 of a method for generating a toxicological ailment nourishment program is illustrated. At step 605, a computing device 104 obtains a toxicological indicator 108. Computing device 104 includes any of the computing device 104 as described above, in reference to FIGS. 1-5. Toxicological indicator 108 includes any of the toxicological indicator 108 as described above, in reference to FIGS. 1-5.

Still referring to FIG. 6, at step 610, computing device 104 identifies a toxicological profile 112 as a function of toxicological indicator 108. Toxicological profile 112 includes any of the toxicological profile 112 as described above, in reference to FIGS. 1-5. Computing device 104 identifies toxicological profile 112 by determining at least a xenobiotic 116 as a function of the toxicological indicator 108. Xenobiotic 116 includes any of the xenobiotic 116 as described above, in reference to FIGS. 1-5. Computing device 104 identifies toxicological profile 112 by obtaining an exposure input 120. Exposure input 120 includes any of the exposure input 120 as described above, in reference to FIGS. 1-5. Computing device 104 identifies toxicological profile 112 as a function of xenobiotic 116 and exposure input 120 using a profile machine-learning model 124. Profile machine-learning model 124 includes any of the profile machine-learning model 124 as described above, in reference to FIGS. 1-5.

Still referring to FIG. 6, at step 615, computing device 104 determines an edible 128 as a function of toxicological profile 112. Edible 128 includes any of the edible 128 as described above, in reference to FIGS. 1-5.

Still referring to FIG. 6, at step 620, computing device 104 generates a nourishment program 132 as a function of edible 128. Nourishment program 132 includes any of the nourishment program 132 as described above, in reference to FIGS. 1-5.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

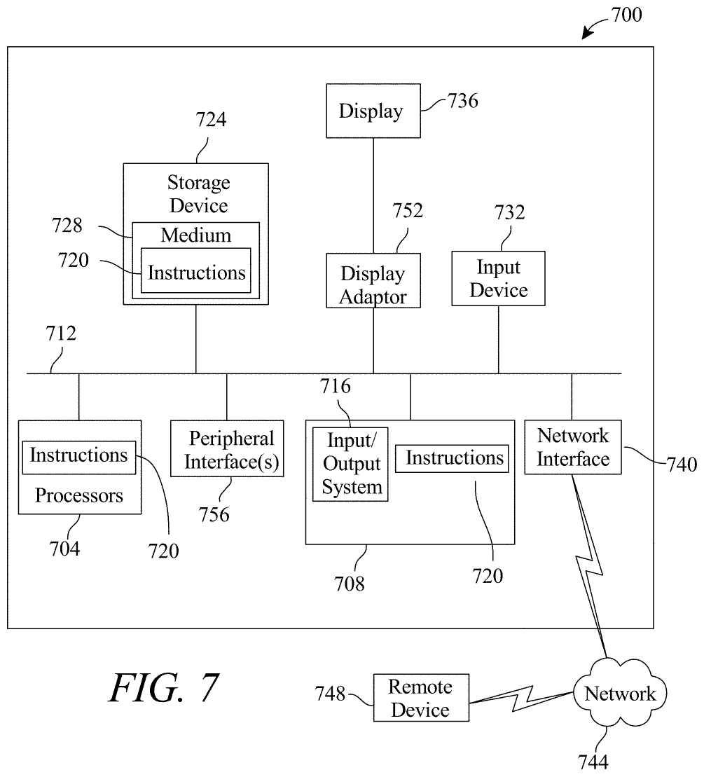

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating a toxicological ailment nourishment program, the system comprising:
a computing device, the computing device configured to:
obtain a toxicological indicator;
identify a toxicological profile as a function of the toxicological indicator, wherein identifying further comprises:
determining at least a xenobiotic as a function of the toxicological indicator;
obtaining an exposure input; and
identifying the toxicological profile as a function of the at least a xenobiotic and the exposure input using a profile machine-learning model;
determine an edible as a function of the toxicological profile; and
generate a nourishment program as a function of the edible.

2. The system of claim 1, wherein obtaining the exposure input further comprises receiving an exposure route and obtaining the exposure input as a function of the exposure route.

3. The system of claim 1, wherein determining the at least a xenobiotic further comprises identifying a toxic range and determining the at least a xenobiotic as a function of the toxic range.

4. The system of claim 1, wherein identifying the toxicological profile further comprises:
receiving a progression element;
determining a toxicity stage as a function of the progression element; and
identifying the toxicological profile as a function of the toxicity stage.

5. The system of claim 1, wherein identifying the toxicological profile further comprises determining a physiological impact and identifying the toxicological profile as a function of the physiological impact.

6. The system of claim 5, wherein determining the physiological impact further comprises:
receiving a binding element from a medical guideline; and
determining the physiological impact as a function of the dosage vector and the binding element using a physiological machine-learning model.

7. The system of claim 1, wherein identifying the toxicological profile includes determining a toxicological ailment and producing the toxicological profile as a function of the toxicological ailment.

8. The system of claim 1, wherein determining the edible further comprises identifying a toxic response vector and determining the edible as a function of the toxic response vector.

9. The system of claim 8, wherein identifying a toxic response vector further comprises:
determining a hormetic element; and
identifying the toxic response vector as a function of the hormetic element and toxicological profile using a response machine-learning model.

10. The system of claim 1, wherein determining the edible further comprises:
obtaining an elimination element; and
determining the edible as a function of the elimination element.

11. A method for generating a toxicological ailment nourishment program, the method comprising:
obtaining, by a computing device, a toxicological indicator;
identifying, by the computing device, a toxicological profile as a function of the toxicological indicator, wherein identifying further comprises:
determining at least a xenobiotic as a function of the toxicological indicator;
obtaining an exposure input; and
identifying the toxicological profile as a function of the at least a xenobiotic and the exposure input using a profile machine-learning model;
determining, by the computing device, an edible as a function of the toxicological profile; and
generating, by the computing device, a nourishment program as a function of the edible.

12. The method of claim 11, wherein obtaining the exposure input further comprises receiving an exposure route and obtaining the exposure input as a function of the exposure route.

13. The method of claim 11, wherein determining the at least a xenobiotic further comprises identifying a toxic range and determining the at least a xenobiotic as a function of the toxic range.

14. The method of claim 11, wherein identifying the toxicological profile further comprises:
receiving a progression element;
determining a toxicity stage as a function of the progression element; and
identifying the toxicological profile as a function of the toxicity stage.

15. The method of claim 11, wherein identifying the toxicological profile further comprises determining a physiological impact and identifying the toxicological profile as a function of the physiological impact.

16. The method of claim 15, wherein determining the physiological impact further comprises:
receiving a binding element from a medical guideline; and
determining the physiological impact as a function of the dosage vector and the binding element using a physiological machine-learning model.

17. The method of claim 11, wherein identifying the toxicological profile includes determining a toxicological ailment and producing the toxicological profile as a function of the toxicological ailment.

18. The method of claim 11, wherein determining the edible further comprises identifying a toxic response vector and determining the edible as a function of the toxic response vector.

19. The method of claim 18, wherein identifying a toxic response vector further comprises:
determining a hormetic element; and
identifying the toxic response vector as a function of the hormetic element and toxicological profile using a response machine-learning model.

20. The method of claim 11, wherein determining the edible further comprises:
obtaining an elimination element; and
determining the edible as a function of the elimination element.
